(19) United States
(12) Patent Application Publication
(10) Pub. No.: US 2023/0360253 A1
(43) Pub. Date: Nov. 9, 2023

(54) MEDICAL INFORMATION PROCESSING SYSTEM AND MEDICAL INFORMATION PROCESSING APPARATUS

(71) Applicant: Canon Medical Systems Corporation, Otawara-shi (JP)

(72) Inventors: Akihito TAKAHASHI, Nasushiobara (JP); Yoshiyasu HAYASHI, Nasushiobara (JP); Junichi YAMAGISHI, Nasushiobara (JP); Yosuke KAYUKAWA, Otawara (JP); Hag Chang LEE, Otawara (JP); Hisayasu YUMIZA, Otawara (JP)

(73) Assignee: Canon Medical Systems Corporation, Otawara-shi (JP)

(21) Appl. No.: 18/306,276

(22) Filed: Apr. 25, 2023

(30) Foreign Application Priority Data
May 9, 2022 (JP) 2022-077111

(51) Int. Cl. G06T 7/70; G06V 20/50; G06V 40/10; G06T 7/20

(52) U.S. Cl. CPC G06T 7/70; G06V 20/50; G06V 40/10; G06T 7/20; G16H 70/20

(57) ABSTRACT

A medical information processing apparatus includes a processing circuit. The processing circuit is configured to: determine an operating area relating to a device having a predetermined movable range, identify positional coordinates corresponding to the operating area in an examination room and positional coordinates of an obstacle in the examination room, based on a camera image taken by an optical camera installed in the examination room; and report a detection result if the positional coordinates of the obstacle are included in the positional coordinates of the operating area.

### CROSS-REFERENCE TO RELATED APPLICATIONS

This application is based upon and claims the benefit of priority from Japanese Patent Application No. 2022-077111, filed May 9, 2022, the entire contents of which are incorporated herein by reference.

### FIELD

Embodiments described herein relate generally to a medical information processing system and a medical information processing apparatus.

### BACKGROUND

In a medical examination room where a medical examination or procedures are conducted using a medical image diagnostic apparatus or the like, various apparatuses are arranged, such as a C-arm or other apparatuses whose operating area (range of motion) is restricted, and apparatuses that can freely move around the examination room. If these apparatuses come into contact (and mutually interfere) during an examination, there is a possibility of causing grave circumstances for a patient and/or medical staff. For this reason, when an operator moves a movable apparatus in the examination room, such an apparatus needs to be placed at a position where it would not interfere with an apparatus with a restricted operating area. In order to avoid the interference, however, it is necessary to rely on the operator's sensory perception and experience when moving the movable apparatus. In addition, depending on the type of an examination and procedure, the operating areas of other apparatuses may vary. It is therefore difficult to avoid interference by depending solely on the operator's sensory perception and experience.

### BRIEF DESCRIPTION OF THE DRAWINGS

FIG. 1 is a diagram for showing an exemplary configuration of a medical information processing system according to the first embodiment.

FIG. 2 is a diagram for showing an exemplary interior arrangement of an examination room according to the first embodiment.

FIG. 3 is a schematic diagram for showing an exemplary arrangement of optical cameras in the examination room according to the first embodiment.

FIG. 4 is a flowchart for showing an exemplary processing procedure of a notification process performed by the medical information processing system according to the first embodiment.

FIG. 5 is a flowchart for showing an exemplary processing procedure of a notification process performed by the medical information processing system according to the first modification of the first embodiment.

FIG. 6 is a flowchart for showing an exemplary processing procedure of a notification process performed by the medical information processing system according to the second modification of the first embodiment.

FIG. 7 is a flowchart for showing an exemplary processing procedure of a notification process performed by the medical information processing system according to the third modification of the first embodiment.

FIG. 8 is a diagram for showing an exemplary configuration of a medical information processing system according to the second embodiment.

FIG. 9 is a diagram for showing an exemplary interior arrangement of an examination room according to the second embodiment.

FIG. 10 is a flowchart for showing an exemplary processing procedure of a notification process performed by the medical information processing system according to the second embodiment.

FIG. 11 is a diagram for showing an exemplary configuration of a medical information processing system according to the fourth embodiment.

FIG. 12 is a flowchart for showing an exemplary processing procedure of a notification process performed by the medical information processing system according to the fourth embodiment.

### DETAILED DESCRIPTION

In general, according to one embodiment, a medical information processing apparatus includes a processing circuit. The processing circuit is configured to: determine an operating area relating to a device having a predetermined movable range, identify positional coordinates corresponding to the operating area in an examination room and positional coordinates of an obstacle in the examination room, based on a camera image taken by an optical camera installed in the examination room; and report a detection result if the positional coordinates of the obstacle are included in the positional coordinates of the operating area.

Embodiments of a medical information processing system and medical information processing apparatus will be described in detail below by referring to the drawings. In the following description, structural elements having substantially the same operations and configurations will be denoted by the same reference symbols, and the explanation thereof will be repeated only where necessary.

### First Embodiment

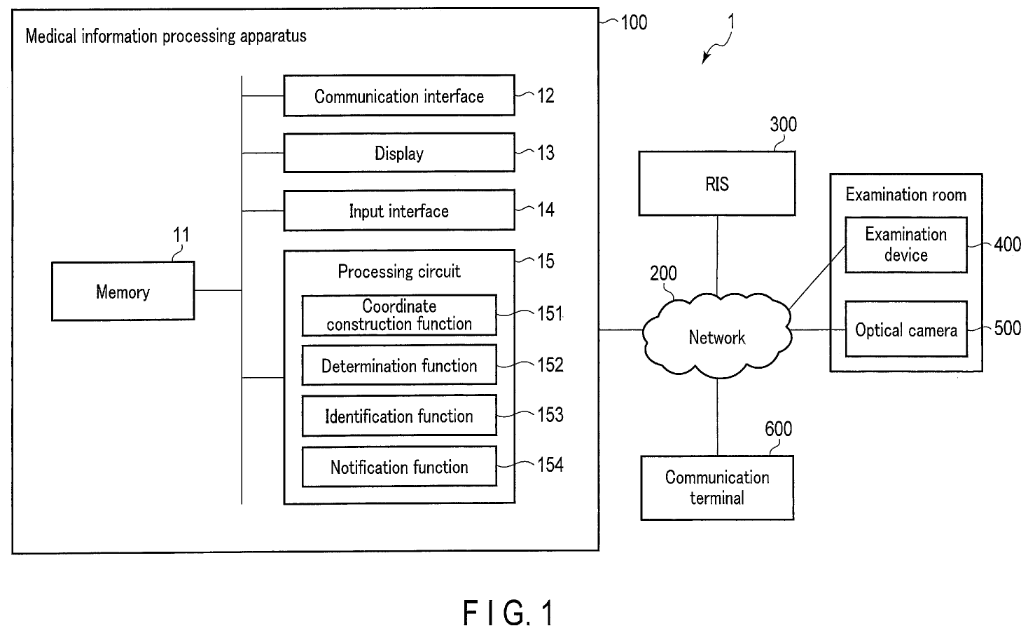

FIG. 1 is a diagram for showing a configuration of a medical information processing system 1. The medical information processing system 1 is an assisting system for medical examinations and procedures conducted according to an examination protocol, using a medical image diagnostic apparatus installed in an examination room. The medical information processing system 1 includes a medical information processing apparatus 100. The medical information processing apparatus 100 may be arranged in an operator's room from which the apparatus in the examination room is handled. Hereinafter, the term “examination” may be interpreted as “procedure”.

The medical information processing apparatus 100 is connected by way of a network 200 to a radiological information system (hereinafter referred to as “RIS”) 300, apparatuses provided in the examination room, and a communication terminal 600 held by the personnel in the examination room. The network 200 may be a local area network (LAN). The connection to the network 200 may be established in either a wired or wireless manner. In addition, the connection is not limited to LAN if security can be ensured by virtual private network (VPN) or the like. The connection may be established to the Internet or other public communication network.

The RIS 300 is a system for managing examination information relating to radiographical examination tasks. The examination information includes examination order information relating to examination orders, and examination implementation information relating to examination results. The RIS 300 accumulates examination order information that has been input by users, adds various types of setting information to the information, and manages the accumulated information as examination reservation information. The examination order information may include information relating to examination protocols and information relating to examinations that are to be conducted. The examination-related information may include information on equipment to be used for the examination and information on a physician conducting the examination. The equipment-related information may include the type, model, size, geometry, and operating area of the equipment. The physician-related information may include the name, department, age, gender, and procedural preferences of a physician. Based on the examination reservation information, the RIS 300 transmits an examination order to a medical image diagnostic apparatus. The RIS 300 also transmits, to the Hospital Information System (HIS) or the like, examination implementation information generated by the medical image diagnostic apparatus upon the implementation of the examination.

Examination devices 400 are arranged in the examination room. The examination devices 400 may include medical image diagnostic apparatuses, monitors, and the like. The medical image diagnostic apparatuses may be of various different imaging modalities for imaging the inside of the body of a patient, such as an X-ray computed tomography apparatus (CT device), a magnetic resonance imaging apparatus (MRI apparatus), an ultrasonic diagnostic apparatus, and an X-ray diagnostic apparatus.

The examination devices 400 in the examination room can be divided into stationary devices whose operating area (range of motion) is limited, and movable devices whose operating area is not limited.

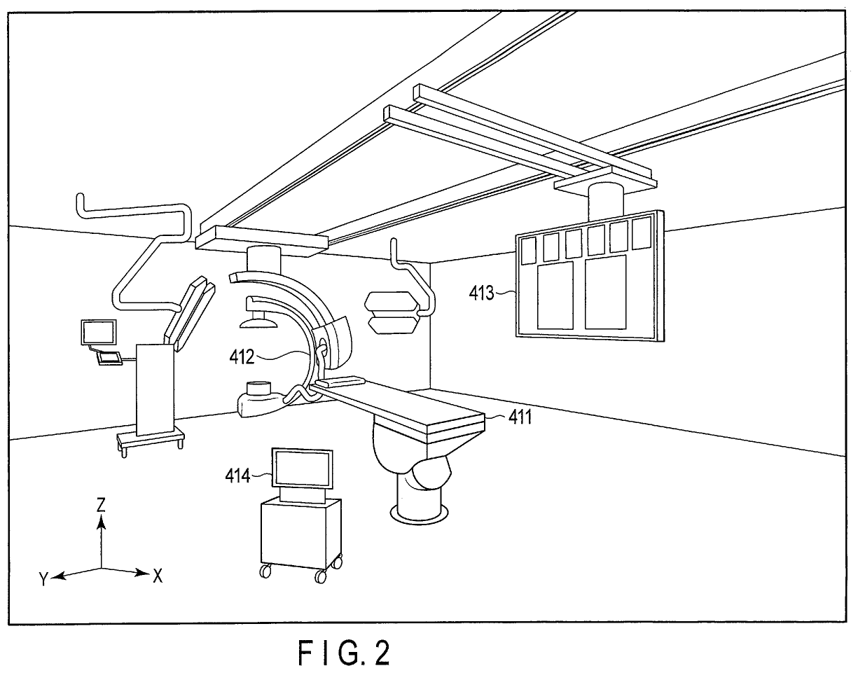

Stationary devices may include electrically powered devices and fixed-trajectory devices. An electrically powered device is electrically operated through external manipulation. The motion trajectory of an electrically powered device at the time of executing an examination protocol is predetermined in accordance with the configuration of the device or settings of the examination protocol. Examples of the examination device 400 categorized as electrically powered devices include a table of a medical image diagnostic apparatus, a supporting device, and a C-arm of an X-ray diagnostic apparatus. FIG. 2 is a diagram for showing an exemplary interior arrangement of an examination room. In the example of FIG. 2, a table 411 on which a patient lies and a C-arm 412 are arranged as electrically powered devices in the examination room.

A fixed-trajectory device is manually operated, with its motion trajectory determined by the structure of the device. The examination devices 400 categorized as fixed-trajectory devices include a monitor hanging from the ceiling and movable along a rail attached to the ceiling. In the example of FIG. 2, as a fixed-trajectory device, a monitor 413 is arranged in the examination room.

A movable device is arranged in the examination room, and is manually moved, with its range of motion unrestricted. The examination devices 400 categorized as movable devices include an ultrasonic diagnostic apparatus freely movable on the floor of the examination room, a stretcher, and an intravenous stand. In the example of FIG. 2, an ultrasonic diagnostic apparatus 414 is arranged as a movable device in the examination room.

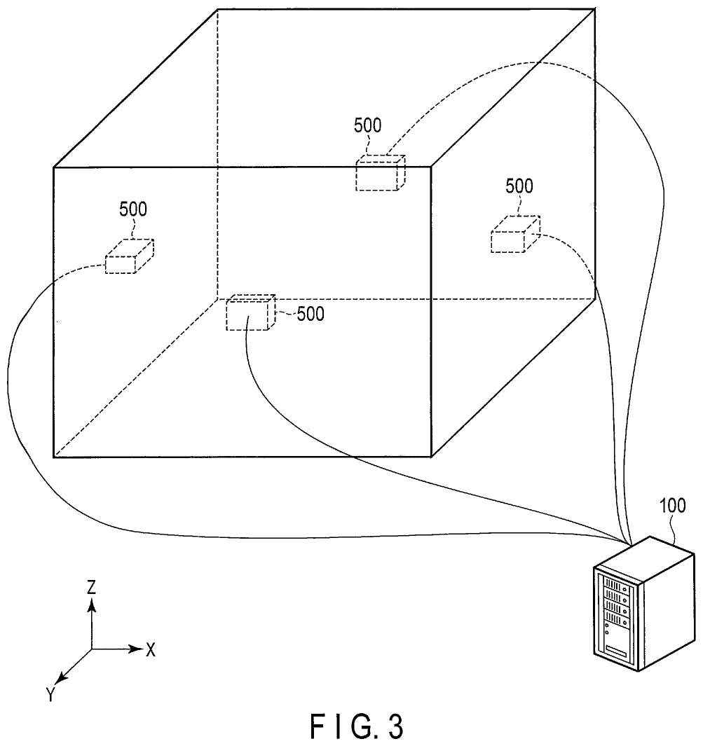

In addition, in order to construct a 3D coordinate space of the examination room, an optical camera 500 is arranged in the examination room. FIG. 3 is a schematic diagram for showing an exemplary arrangement of an examination room with optical cameras 500. As an optical camera 500, a commonly used camera that can take moving images is adopted. The number of optical cameras 500 arranged can be determined as needed to construct the 3D coordinate space. For instance, four optical cameras 500 may be respectively arranged on the four walls of the examination room. In the example of FIG. 3, four optical cameras 500 are provided in the examination room. Each optical camera 500 is arranged so as to take images of the interior of the examination room. The optical cameras 500 are connected to the medical information processing apparatus 100 via a network 200 so that the obtained camera images can be sent to the medical information processing apparatus 100.

A communication terminal 600 is a terminal device that is externally communicable and carried by each medical worker in the examination room. The communication terminal 600 is connected to the medical information processing apparatus 100 via the network 200, and is communicable with the medical information processing apparatus 100. The communication terminal 600 receives an alert signal from the medical information processing apparatus 100, and provides a notification to the medical worker who carries the communication terminal 600. The communication terminal 600 may be a display terminal that includes a display to show the descriptions of the received signal in an image or text. Alternatively, the communication terminal 600 may be a vibrator that vibrates upon reception of an external signal, or a voice communication device that reports the description of the received signal by voice. The communication terminal 600 may be of a type wearable on the wrist of a medical worker.

The medical information processing apparatus 100 may include a memory 11, a communication interface 12, a display 13, an input interface 14, and a processing circuit 15. In the description below, the medical information processing apparatus 100 is explained as implementing multiple functions on a single machine. The functions, however, may be implemented on different machines. The functions implemented by the medical information processing apparatus 100 may be distributed to different console devices or work stations. The medical information processing apparatus 100 may not include a communication interface 12, a display 13 or an input interface 14, but may include a memory 11 and a processing circuit 15 only. FIG. 3 shows a medical information processing apparatus 100 without a display 13 or an input interface 14.

The memory 11 is a storage device, such as a hard disk drive (HDD), a solid state drive (SSD), and an integrated circuit, configured to store various types of information. In addition to an HDD and SSD, the memory 11 can be a portable storage medium such as a compact disc (CD), a digital versatile disc (DVD), and a flash memory. The memory 11 may be a drive that allows for reading and writing of information of various types from and to a semiconductor memory element such as a flash memory and a random access memory (RAM). The storage region of the memory 11 may be provided in the medical information processing apparatus 100, or in an external storage device connected by way of a network.

The memory 11 is configured to store programs to be executed by the processing circuit 15, various types of data to be used in the processing performed by the processing circuit 15, and the like. Such programs may include a program that is installed in advance in a computer through a network or from a non-transitory computer-readable storage medium to cause the computer to realize various functions of the processing circuit 15. The data discussed throughout this specification is digital data in general. The memory 11 is an example of a storage unit.

The communication interface 12 is a network interface configured to control communications among the RIS 300, examination devices 400, optical cameras 500, and communication terminals 600 as well as other external devices via the network 200.

The display 13 is configured to display various types of information. For instance, the display 13 outputs medical information generated by the processing circuit 15, a graphical user interface (GUI) for receiving various operations from the operator, and the like. The display 13 may be a liquid crystal display or a cathode ray tube (CRT) display. The display 13 is an example of a display unit.

The input interface 14 receives various input operations from an operator, converts the received input operations to electric signals, and outputs the signals to the processing circuit 15. For instance, the interface 14 receives inputs of medical information, inputs of various command signals from the operator, and the like. The input interface 14 is realized by a mouse, a keyboard, a trackball, switch buttons, a touch screen in which a display screen and a touch pad are integrated, a non-contact input circuit adopting optical sensors, a voice input circuit, and the like for performing various processes of the processing circuit 15. The input interface is connected to the processing circuit 15 so that the input operation received from the operation can be converted to an electric signal and output to the control circuit. Throughout this specification, the input interface is not limited to a physical operational component such as a mouse and a keyboard. For instance, examples of the input interface may include an electric signal processing circuit configured to receive an electric signal corresponding to an input operation from an external input device provided separately from the present apparatus, and output this electric signal to the processing circuit 15. The input interface 14 is an example of an input unit.

The processing circuit 15 is configured to control the overall operation of the medical information processing apparatus 100. The processing circuit 15 is a processor configured to, upon calling and executing a program from the memory 11, implement a coordinate construction function 151, a determination function 152, an identification function 153, and a notification function 154. The processing circuit 15 that realizes each of the coordinate construction function 151, determination function 152, identification function 153, and notification function 154 is an example of a coordinate construction unit, a determination unit, an identification unit, and a notification unit, respectively.

In the description of FIG. 1, it is a single processing circuit 15 that realizes the coordinate construction function 151, determination function 152, identification function 153, and notification function 154. These functions, however, may be realized by combining multiple independent processors to form a processing circuit and causing these processors to execute the program. Furthermore, the coordinate construction function 151, determination function 152, identification function 153, and notification function 154 may be implemented as individual hardware circuits. The above description of the functions executed by the processing circuit 15 applies to the embodiments and modification examples discussed below.

In the description of the medical information processing apparatus 100, multiple functions are implemented with a single console. These functions, however, may be implemented by different devices. For instance, the functions of the processing circuit 15 may be distributed over different devices. The functions of the processing circuit 15 of the medical information processing apparatus 100 may be installed in a medical image diagnostic apparatus in the examination room or a control device or the like in the operator's room.

The term “processor” used in the above description may denote a central processing unit (CPU), a graphics processing unit (GPU), an application specific integrated circuit (ASIC), a programmable logic device (e.g., a simple programmable logic device (SPLD), a complex programmable logic device (CPLD), and a field programmable gate array (FPGA)), or the like. For instance, if the processor is a CPU, the processor reads and executes a program stored in the memory circuitry to realize the functions. If the processor is an ASIC, instead of storing the program in the memory circuitry, the functions are directly installed in the circuitry of the processor as a logic circuit. The processors according to the embodiments are not limited to a single circuit for each processor, but may be configured as a single processor by combining different independent circuits to realize the functions. Furthermore, the structural components illustrated in FIG. 1 may be integrated into one processor to realize their functions. The above description of the processor applies to other embodiments and modification examples.

With the coordinate construction function 151, the processing circuit 15 constitutes 3D space coordinates of the examination room using camera images obtained by the optical cameras 500. The processing circuit 15 realizing the coordinate construction function 151 is an example of a coordinate construction unit. The processing circuit 15 constructs the 3D space coordinates of the entire examination room, for example, by defining a mutually perpendicular X-axis and Y-axis along the horizontal direction, and Z-axis perpendicular to the X-axis and Y-axis along the vertical direction, as illustrated in FIG. 2. To construct the 3D space coordinates, a well-known 3D space recognition method using camera images taken by one or more cameras can be adopted.

With the determination function 152, the processing circuit 15 determines the operating area of a stationary device whose operable range (range of motion) is predetermined. Here, the processing circuit 15 determines the operating area of each of the stationary devices installed in the examination room. The operating area denotes a 3D area to which the stationary device may possibly reach with a movement of the device during an examination. The processing circuit 15 that realizes the determination function 152 is an example of the determination unit.

To determine the operating area of a specific stationary device, the processing circuit 15 first acquires from the RIS 300 the device information relating to this stationary device. The device information that is to be acquired may include the size, geometry, type, model and the like of the stationary device. Based on the acquired device information, the processing circuit 15 determines the operating area of the stationary device according to an examination protocol that is to be executed. The operating area is determined to correspond to the size and geometry of the stationary device. The processing circuit 15 stores the determined operating area in the memory 11.

The operating area defines an area to which the stationary device may reach during an examination. For this reason, if a movable device is left unattended within the operating area, it may contact the moving stationary device. The operating area is therefore a region of the stationary device in which there is a possibility of interference with an obstacle. Obstacles include a person present in the examination room and movable devices that freely move around the examination room. A person in the examination room may be a medical worker such as an operator of the movable device, a physician, and a clinical technician. Hereinafter, the operating area may also be referred to as a range of motion or an interference area.

With the identification function 153, the processing circuit 15 is configured to identify the positional coordinates corresponding to the operating area of the examination room and the positional coordinates of an obstacle in the examination room, using the camera images taken with the optical cameras 500 installed in the examination room. The processing circuit 15 that realizes the identification function 153 is an example of the identification unit.

To identify the positional coordinates of the operating area, the processing circuit 15 applies the 3D space coordinates constructed with the coordinate construction function 151 to the operating area determined with the determination function 152. In this manner, the positional coordinates of the operating area in the examination room can be identified.

To identify the positional coordinates of an obstacle, the processing circuit 15 detects the obstacle from the camera images obtained by the optical cameras 500. For the method of detecting an obstacle from camera images, a well-known image recognition technique may be adopted. For instance, with the well-known image recognition technique, an object that is not registered as a stationary device can be detected as an obstacle. Alternatively, an object that is moving within the examination room is detected, and is compared with the movements of the registered stationary devices. If the movement of the object does not correspond to the movements of any stationary device, the detected object is determined as an obstacle. Furthermore, with the well-known image recognition technique, whether or not the object detected as an obstacle is a person may be determined. If the object detected as an obstacle is determined as a person with the well-known image recognition technique, the detected person may be identified by referring to the preregistered physical attribute information or the like. Furthermore, with the well-known image recognition technique, the device type of the detected object may be identified by referring to the geometry or the like of the preregistered device. After detection of the obstacle, the processing circuit 15 identifies the positional coordinates of the obstacle in the examination room by applying the detected obstacle to the 3D space coordinates constructed with the coordinate construction function 151.

With the notification function 154, the processing circuit 15 reports a detection result if the positional coordinates of an obstacle are included in the positional coordinates of the operating area.

In particular, the processing circuit 15 compares the positional coordinates of the obstacle identified with the identification function 153 with the positional coordinates of the operating area in real time to determine whether or not the obstacle is located in the operating area. If the obstacle is within the operating area, the processing circuit 15 informs the people in the examination room. For instance, if at least one set of the positional coordinates of the obstacle match the positional coordinates of the operating area, it is determined that the obstacle is located within the operating area. Alternatively, if a certain proportion or more of the entire series of the positional coordinates of the obstacle is included in the positional coordinates of the operating area, it is determined that the obstacle is located within the operating area.

For notification, methods such as sounding an alert, reporting a risk of interference by voice, and indicating the risk on the monitor in the examination room may be adopted. The processing circuit 15 that realizes the notification function 154 is an example of the notification unit.

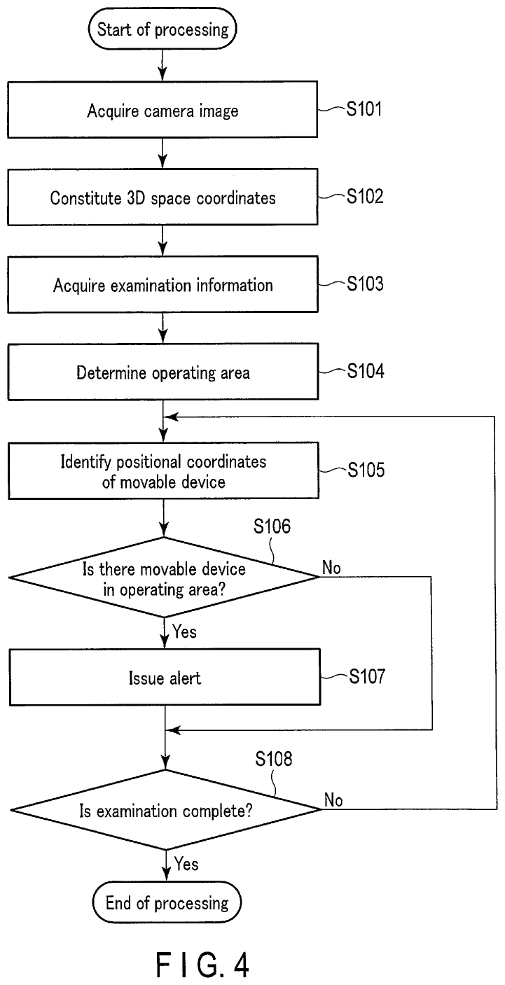

Next, the notification processing operation executed by the medical information processing system 1 will be described. In the notification process, camera images taken by the optical cameras 500 are acquired in real time so that an alert can be issued when an obstacle is about to be brought into and placed in the interference area during an examination. FIG. 4 is a flowchart showing an exemplary procedure of the notification process. The processing procedure of the operations indicated below is presented merely as an example, and the respective operations may be suitably changed as needed. An omission, replacement and addition of steps may be made to the processing procedure described below according to the embodiment.

### Notification Process

### Step S101

The processing circuit 15 obtains camera images of the interior of the examination room from the optical cameras 500.

### Step S102

Next, with the coordinate construction function 151, the processing circuit 15 constitutes 3D space coordinates of the entire examination room based on the obtained camera images.

### Step S103

With the determination function 152, the processing circuit 15 acquires examination information relating to a planned examination from the RIS 300. In this manner, the processing circuit 15 acquires the operating area of a stationary device to be used for the examination and information relating to the operations of this stationary device during the examination.

### Step S104

Thereafter, with the determination function 152, the processing circuit 15 determines the operating area of the stationary device based on the acquired examination information, and with the identification function 153, the processing circuit 15 identifies the positional coordinates of the operating area of the stationary device on the 3D space coordinates. For instance, the trajectory of the C-arm becomes the operating area of the C-arm. In each examination protocol, the movement of the C-arm is predetermined, and therefore the operating area of the C-arm can be computed from the acquired information relating to the size and geometry of the C-arm and information relating to the operation of the C-arm for a planned examination protocol.

### Step S105

Next, with the identification function 153, the processing circuit 15 performs predetermined image processing upon the camera images continuously acquired from the optical cameras 500, thereby detecting any obstacle placed in the examination room and identifying the positional coordinates of the detected obstacle on the 3D space coordinates.

### Step S106

With the identification function 153, the processing circuit 15 determines whether or not the identified positional coordinates of the obstacle are included in the operating area of the stationary device. If there is more than one stationary device for which the determination of the operating area is conducted, it is determined whether or not the identified positional coordinates of the obstacle are included in at least one of the operating areas.

### Step S107

If the positional coordinates of the obstacle are included in the positional coordinates of the operating area of the stationary device (yes at step S106), the processing circuit 15 reports, with the notification function 154, that an obstacle is placed in the operating area of the stationary device. For instance, the processing circuit 15 sends a signal to a control device configured to control a loudspeaker installed in the examination room so that an alert sound or alert message can be issued through the loudspeaker. Alternatively, an alert message may be displayed on the monitor in the examination room.

### Step S108

If the positional coordinates of the obstacle are not included in the positional coordinates of the operating area of the stationary device (no at step S106), or after the processing of step S107 is conducted to report that an obstacle is within the operating area of the stationary device, the processing circuit 15 acquires the examination progress information and information relating to imaging by the medical image diagnostic apparatus, and determines whether or not the examination is complete based on the acquired information.

The processing circuit 15 repeats the process of steps S105 through S107 until the examination is complete. In this manner, every time a new camera image is acquired, the processing circuit 15 identifies the positional coordinates of an obstacle and issues an alert to the people in the examination room if the obstacle is placed within the operating area of the stationary device. Upon removal of the obstacle, which has been in the operating area of the stationary device, from the operating area, the processing circuit 15 terminates the alerting.

Upon the completion of the examination (yes at step S108), the processing circuit 15 terminates the notification process.

The effects of the medical information processing system 1 according to the present embodiment will be described below.

The medical information processing apparatus 100 in the medical information processing system 1 according to the present embodiment is configured to determine the operating area of a device whose operable range (range of motion) is predetermined; identify the positional coordinates corresponding to the operating area in the examination room and the positional coordinates of an obstacle in the examination room based on the camera images taken by the optical camera 500 in the examination room; and report a detection result if the positional coordinates of the obstacle are included in the positional coordinates of the operating area.

An obstacle may be a person present in the examination room and a movable device that can freely move around the examination room. A stationary device may be a device electrically operated under external control such as a table and a C-arm, or a device with a restricted movable range such as a monitor.

With the above configuration, the medical information processing apparatus 100 of the medical information processing system 1 according to the present embodiment can construct coordinate axis information for the entire examination room, using the camera images obtained by optical cameras 500 installed in the examination room, and can acquire the positional information of an obstacle such as a movable device. In addition, the medical information processing apparatus 100 can inform people present in the examination room that there is a possibility of an interference of the stationary device with an obstacle when the obstacle is placed during the implementation of a medical examination or procedure within the operating area (interference area) of the stationary device whose motion trajectory has been determined. For instance, if the ultrasonic diagnostic apparatus 414 illustrated in FIG. 2 is detected as being placed within the movable range of the table 411, an alert is issued by voice to an operator of the ultrasonic diagnostic apparatus 414 to urge the operator to take an action in order to avoid an interference. The operator, who acknowledges the alert when placing a movable device on the motion trajectory of another device, can easily move and place the movable device at a position that would not cause an interference. Alternatively, an obstacle being within the movable range of the stationary device may be reported to any worker in the examination room so that the person who has acknowledged this notification can remove the obstacle. Such a configuration can prevent an accidental contact. In addition, if there is no alert, a movable device can be placed safely at an originally intended position. As discussed above, the medical information processing apparatus 100 of the medical information processing system 1 according to the present embodiment contributes to realization of a swift and safe examination and medical procedure.

An obstacle may be a medical worker present in the examination room. In this case, when anyone in the examination room moves into the operating area of the stationary device, an alert can be issued to this person. The alerted worker can move out of the area until the alerting is stopped so that the person can stay at a position where no contact will occur with the stationary device. This configuration can prevent an accidental contact.

An obstacle may be an electrically powered device that is not equipped with an interference controlling function. For instance, for a medical image diagnostic apparatus of a conventional model not equipped with an interference controlling function, its movable portion is detected from camera images to identify the positional coordinates thereof so that, when the movable portion moves into the operating area of another electrically powered device, an alert can be issued.

### First Modification Example of First Embodiment

The first modification example of the first embodiment will be described. In this modification example, the configuration of the first embodiment is modified as indicated below. The medical information processing system 1 and medical information processing apparatus 100 according to this modification example issues an alert only when an obstacle is staying still in the operating area of the stationary device. Parts of the structure, operation and effects that are the same as in the first embodiment will be omitted from the description.

In this modification example, with the notification function 154, the processing circuit 15 determines the moving state of an obstacle based on the camera images, and reports the detection result when the obstacle is staying still and the positional coordinates of the obstacle are included in the positional coordinates of the operating area.

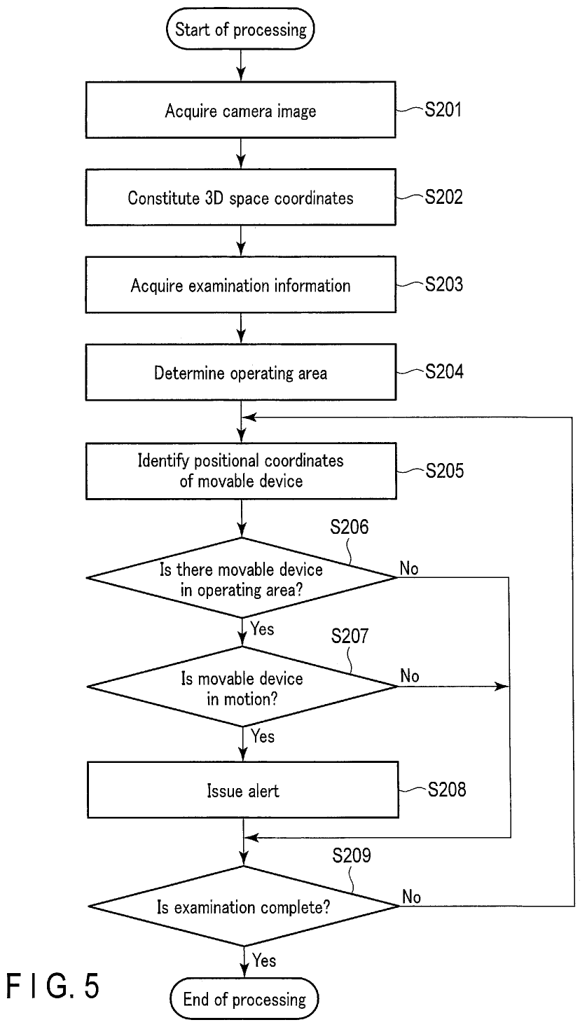

Next, the notification processing operation executed by the medical information processing system 1 will be described. FIG. 5 is a flowchart for showing an exemplary procedure of the notification process performed by the processing circuit 15 according to the present modification example. The operations at steps S201 to S206 and S208 to S209 are the same as those at steps S101 to S108 in FIG. 4, and the explanation thereof is omitted.

### Image Generation Process

### Step S207

If the positional coordinates of a movable device are included in the positional coordinates of the operating area of the stationary device (yes at step S206), with the notification function 154, the processing circuit 15 determines whether or not the movable device is in motion. For this, the processing circuit 15 may continuously acquire the positional coordinates of the movable device identified from the camera images, and determine that the movable device is staying still if the length of time of the positional coordinates being unchanged is longer than a predetermined time length. If not, the processing circuit 15 determines that the movable device is in motion. If the movable device is in motion, the process proceeds to step S209, without reporting that the movable device is in the operating area of the stationary device.

### Step S208

If the movable device is staying still (yes at step S207), with the notification function 154, the processing circuit 15 reports that the movable device is placed within the operating area of the stationary device.

The effects of the medical information processing system 1 and medical information processing apparatus 100 according to the present modification example will be described below.

If a movable device is left in the operating area (interference area) of the stationary device, there is a risk that the devices may interfere with each other. On the other hand, the movable device passing through the operating area (interference area) of the stationary device will not cause any problem. The medical information processing apparatus 100 of the medical information processing system 1 according to the present modification example determines the moving state of the obstacle based on the camera images and reports the detection result only when the obstacle is staying still and the positional coordinates of the obstacle are included in the positional coordinates of the operating area. With such a configuration, an alert can be issued only when the movable device stays in the operating area of the stationary device for a predetermined length of time or longer.

Even when the obstacle is in motion, a possible risk that may arise if it stays at a present position may be reported by voice or the like. In this case, the processing circuit 15 reports, with the notification function 154, that a possible risk may arise if the obstacle stays at a present position when the obstacle is in motion and the positional coordinates of the obstacle are included in the positional coordinates of the operating area. In this manner, it is possible to call attention to the moving obstacle that is currently within the operating area of the stationary device.

### Second Modification Example of First Embodiment

The second modification example of the first embodiment will be described. In this modification example, the configuration of the first embodiment is modified as indicated below. The medical information processing system 1 and medical information processing apparatus 100 according to the present modification example identify the person staying the closest to the movable device placed in the interference area, and issue an alert to this person. Parts of the structure, operation and effects that are the same as in the first embodiment will be omitted from the description.

According to the present modification example, with the identification function 153, the processing circuit 15 identifies the positional coordinates of people in the examination room. As a method for identifying the positional coordinates of the people in the examination room, the positional coordinates of each person may be calculated based on the signals transmitted from the communication terminals 600 provided individually for the medical staff in the examination room. Alternatively, a well-known human detection process may be adopted and performed upon the camera images acquired from the optical camera 500 so that the people in the examination room may be identified by referring to the pre-registered personal information.

If a movable device is detected as an obstacle and the positional coordinates of the movable device are included in the positional coordinates of the operating area, with the notification function 154, the processing circuit 15 informs the person closest to the movable device of the detection result. For instance, using the positional coordinates of each individual and the positional coordinates of the movable device identified with the identification function 153, the processing circuit 15 identifies the person closest to the movable device in the operating area of the stationary device and issues an alert to this person. The processing circuit 15 may send an alert message to the communication terminal 600 held by the person closest to the movable device. Alternatively, the name of the identified person and the description of the alert may be displayed on the monitor in the examination room.

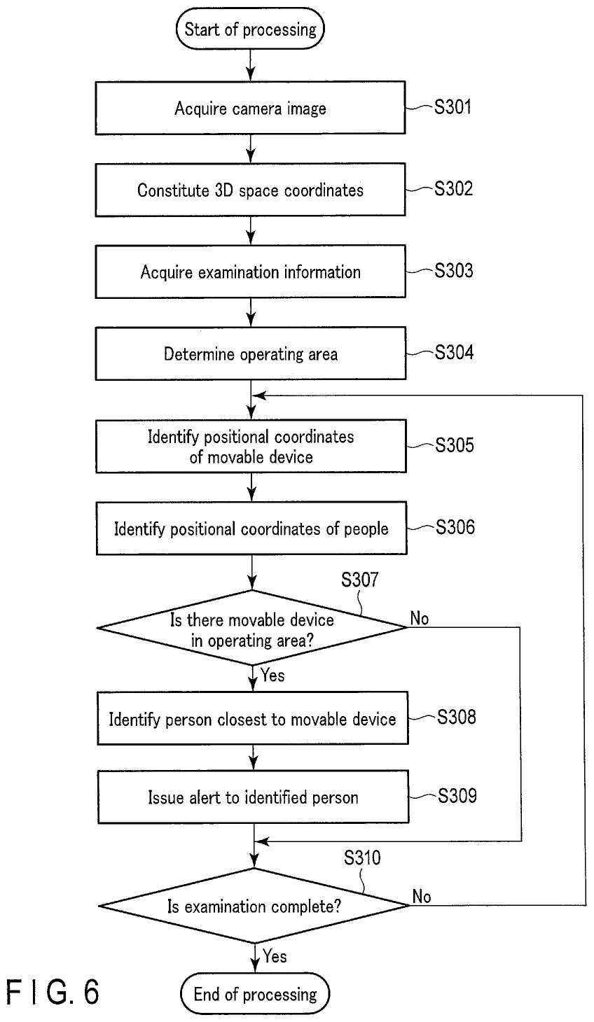

Next, the notification processing operation executed by the medical information processing system 1 will be described. FIG. 6 is a flowchart for showing an exemplary procedure of the notification process performed by the processing circuit 15 according to the present modification example. The operations at steps S301 to S305 and S307 are the same as the operations at step S101 to S106 in FIG. 4, and the explanation thereof is omitted.

### Image Generation Process

### Step S306

After the operation at step S305 of identifying the positional coordinates of the movable device, with the identification function 153, the processing circuit 15 detects people in the examination room and identifies the positional coordinates of each individual.

### Step S308

If the positional coordinates of the movable device are included in the positional coordinates of the operating area of the stationary device (yes at step S207), the processing circuit 15 identifies the person closest to the movable device in the operating area of the stationary device.

### Step S309

With the notification function 154, the processing circuit 15 issues an alert to the identified person.

The effects of the medical information processing system 1 and medical information processing apparatus 100 according to the present modification example will be described below.

The medical information processing apparatus 100 of the medical information processing system 1 according to the present modification example identifies the positional coordinates of the people in the examination room, detects as an obstacle a movable device that freely moves around the examination room, and informs a person close to the movable device of the detection result if the positional coordinates of the movable device are included in the positional coordinates of the operating area. With such a configuration, a notification urging moving of the movable device can be sent, for example to a medical worker present at a position from which the movable device can be easily moved. Even if an electrically powered device that is not equipped with an interference controlling function is detected as an obstacle, a detection result can be sent in a similar manner to the person closest to this electrically powered device.

### Third Modification Example of First Embodiment

The third modification example of the first embodiment will be described. In this modification example, the configuration of the first embodiment is modified as indicated below. In the medical information processing system 1 and medical information processing apparatus 100 according to this modification example, movable devices provided in the examination room are registered in advance, and if a movable device is brought into the interference area, the type of the movable device to be removed from the interference area is reported. Parts of the structure, operation and effects that are the same as in the first embodiment will be omitted from the description.

In this modification example, information regarding obstacles in the examination room is stored in the memory 11. The obstacle information may relate to movable devices. The information relating to a movable device may include the color, type, size, geometry and the like of the movable device.

With the identification function 153, the processing circuit 15 identifies the identification information of an obstacle based on the obstacle information stored in the memory 11. For the identification information, for example the type and model of the movable device may be used as the name of the device that can distinguish the device from other devices in the examination room. For instance, the processing circuit 15 identifies the geometry, color and size of a movable device detected from camera images, and compares such information with the prestored information of the movable devices, so that the type of the detected movable device can be identified.

Furthermore, with the notification function 154, the processing circuit 15 reports the identification information of the obstacle in addition to the detection result upon detection of the positional coordinates of the obstacle being included in the positional coordinates of the operating area. For instance, the type of the movable device placed in the operating area of the stationary device is reported across the examination room.

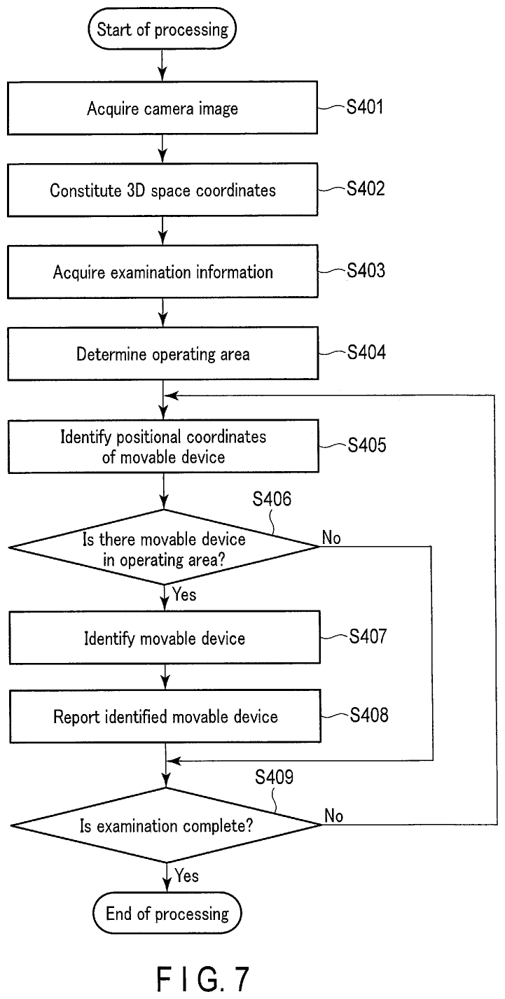

Next, the notification processing operation executed by the medical information processing system 1 will be described. FIG. 7 is a flowchart for showing an exemplary procedure of the notification process executed by the processing circuit 15 according to the present modification example. The operations at steps S401 to S406, S409 are the same respectively as the operations at steps S101 to S106 and S108 in FIG. 4, and the explanation thereof is omitted.

### Image Generation Process

### Step S407

If the positional coordinates of the movable device are included in the positional coordinates of the operating area of the stationary device (yes at step S406), with the identification function 153, the processing circuit 15 identifies the type of the detected movable device through the comparison of the geometry, color and size of the detected movable device with the stored information.

### Step S408

With the notification function 154, the processing circuit 15 reports the information relating to the identified movable device across the examination room. For instance, a voice message may be issued through a loudspeaker installed in the examination room so that the type or serial number of the movable device in the operating area of the stationary device can be reported to the people present in the examination room.

The effects of the medical information processing system 1 and medical information processing apparatus 100 according to the present modification example will be described below.

The medical information processing apparatus 100 of the medical information processing system 1 according to the present modification example can identify the identification information of an obstacle based on the obstacle information prestored in the memory 11, and if the positional coordinates of the obstacle are included in the positional coordinates of the operating area, the medical information processing apparatus 100 reports the identification information of the obstacle in addition to the detection results. With such a configuration, the medical information processing apparatus 100 is capable of reporting specifically which one of the movable devices in the examination room is currently placed in the operating area of the stationary device. The medical worker in the examination room can immediately move the reported device, as a result of which the risk of causing interference between devices can be reliably prevented.

### Second Embodiment

The second embodiment will be described. In the second embodiment, the configuration of the first embodiment is modified as indicated below. According to this embodiment, the operating area of a stationary device is projected onto the floor surface of the examination room or the like. Parts of the structure, operation and effects that are the same as in the first embodiment will be omitted from the description.

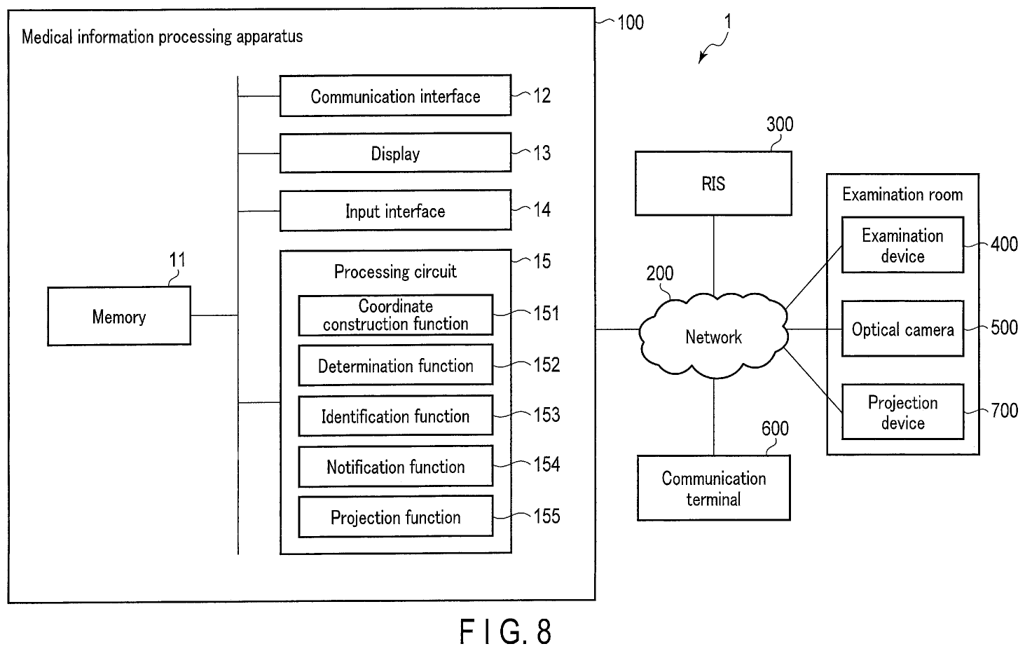

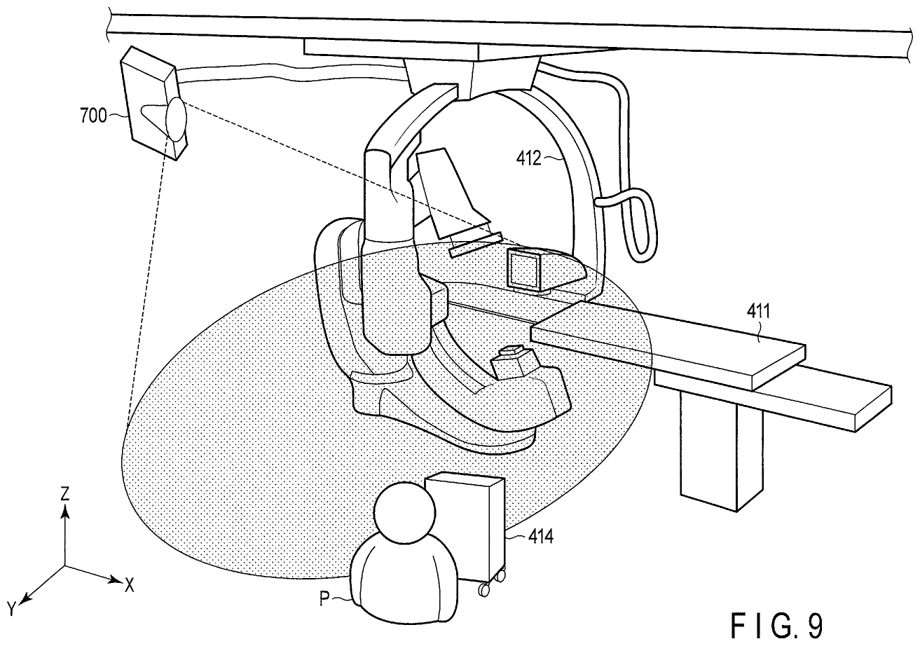

FIG. 8 is a diagram for showing the configuration of a medical information processing system 1 according to the present embodiment. FIG. 9 is a diagram for showing an exemplary interior arrangement of an examination room according to the present embodiment. According to the present embodiment, a projection device 700 for projecting the operating area of a stationary device inside the examination room is installed in the examination room. As the projection device 700, a projector attached to the ceiling of the examination room or to the medical image diagnostic apparatus and projecting an interference area onto the floor of the examination room may be adopted. In the example of FIG. 9, the table 411 and C-arm 412, which are electrically powered devices, the ultrasonic diagnostic apparatus 414, which is a movable device, and the operator P of the ultrasonic diagnostic apparatus 414 are present in the examination room.

The projection device 700 throws projection light for an interference area having 3D space coordinates of a geometry (hereinafter referred to as a “projection geometry”), for example, in a vertical direction onto the floor surface of the examination room. The projection light to be projected may be of a simple shape such as a circle or a rectangle that covers the projection geometry. The projection device 700 may be constituted by a plurality of projectors so that the projection light from these projectors can be combined to create a projection light for a complicated projection geometry.

The processing circuit 15 executes the projection function 155 in addition to the functions described in the first embodiment. With the projection function 155, the processing circuit 15 causes the projection device 700 installed in the examination room to project the operating area of the stationary device identified with the identification function 153.

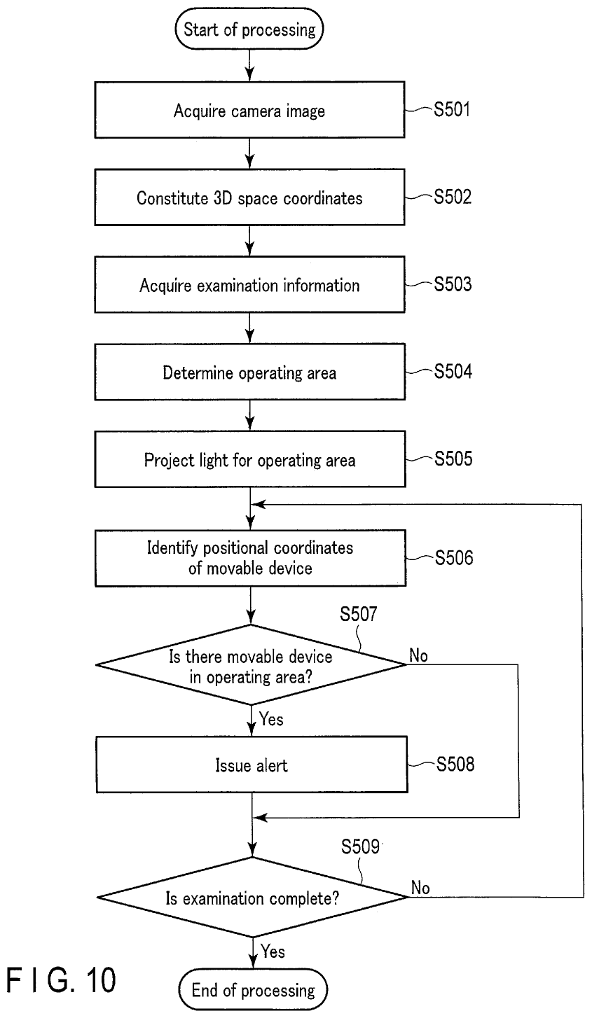

Next, the notification processing operation executed by the medical information processing system 1 will be described. FIG. 10 is a flowchart for showing an exemplary procedure of the notification process executed by the processing circuit 15 according to the present embodiment. The operations at steps S501 to S504 and S506 to S509 are the same as those at steps S101 to S108 in FIG. 4, and the explanation thereof is omitted.

### Image Generation Process

### Step S505

After the process at step S504 in which the operating area of the stationary device is identified, the processing circuit 15 controls the projection device 700 with the projection function 155 such that projection light having a geometry of the operating area of the stationary device when being cast on the floor can be projected onto the floor surface of the examination room. This projection is continued until the completion of the examination.

The effects of the medical information processing system 1 and medical information processing apparatus 100 according to the present embodiment will be described below.

According to the present embodiment, if a movable device is placed in the operating area of the stationary device, a movable device is detected based on the camera images; and if this movable device is identified as being in the operating area of the stationary device, an alert is issued across the examination room. Furthermore, the medical information processing apparatus 100 of the medical information processing system 1 according to the present embodiment causes the projection device 700 installed in the examination room to project the operating area of the stationary device. With such a configuration, the projection geometry of the operating area of the stationary device is projected onto the floor of the examination room until the examination is completed. When moving a movable device in the examination room, the operator of the movable device can avoid the area projected on the floor for the placement of the movable device. Thus, the movable device can be easily arranged while avoiding the operating area of the stationary device. In other words, the present embodiment urges the operator to arrange the movable device at a position out of the interference area.

Instead of projection of the operating area onto the floor surface, the augmented reality (AR) technique may be adopted so as to allow the operator to visually recognize the operating area. In this case, the operator may wear AR glasses. With the AR glasses on, a virtual interference area is superimposed upon the real space of the examination room. Furthermore, with the operating area of the stationary device displayed on the 3D space using the AR technique, even when moving a movable device that is freely movable around the space of the examination room such as a device hanging from the ceiling or the like, the device can still be effectively prevented from interfering with other devices.

### Third Embodiment

The third embodiment will be described. In this embodiment, the configuration of the first embodiment is modified as indicated below. According to the present embodiment, the operating area can be changed in accordance with the type of the medical examination or procedure. Parts of the structure, operation and effects that are the same as in the first embodiment will be omitted from the description.

With the determination function 152, the processing circuit 15 determines the operating area of the stationary device in accordance with the examination protocol. For instance, in order to determine the interference area of an electrically powered device according to a target examination protocol, the processing circuit 15 first acquires the device information of the electrically powered device, and also the examination information of the target examination protocol from the RIS 300. The acquired examination information may include information relating to the operation of the electrically powered device predetermined for the target examination protocol and an operation that may possibly be performed upon the manipulation by the operator under the examination protocol. Based on the acquired device information and examination information, the processing circuit 15 determines the interference area of the electrically powered device according to the target examination protocol.

For instance, the operating area of the stationary device is determined in accordance with a target site of the body to be examined. For instance, the ranges of motion of the table and C-arm during an examination differ between a cardiac examination and abdominal examination. For this reason, the interference area can be set to the minimal area by setting only the ranges of motion of the table and C-arm during a cardiac examination, for example, to an interference area.

In a medical examination or procedure using a biplane X-ray diagnostic apparatus equipped with two arms, X-ray imaging may use only one arm. In such a case, the movable range of only an arm to be used may be set to the operating area of the X-ray diagnostic apparatus so that the minimal area can be defined as the interference area.

An increase in the operating area will reduce the area for arranging movable devices in the examination room, which will make the use of the movable devices inconvenient. According to the medical information processing apparatus 100 of the medical information processing system 1 according to the present embodiment, the interference area is prevented from being defined to be unnecessarily large, and therefore can be set to be minimal. In this manner, a sufficient space can be ensured for the operator to move the movable devices around.

### Modification Example of Third Embodiment

The predispositions and preferences in X-ray imaging angles differ depending on individual operators, as a result of which the range of motion of the C-arm during a medical examination or procedure differs depending on individual operators. Such predispositions and preferences may be stored in advance so that the settings of the operating area can be changed in accordance with the operator's predispositions. In this case, with the determination function 152, the processing circuit 15 determines the operating area in accordance with the operator's operating predispositions. As an operator's predispositions, his/her preferred angle of the C-arm, for example, may be stored in advance. With variations in the operating area in accordance with the operator's predispositions and preferences, the interference area can be set to be minimal. As a result, a larger space can be ensured for the operator to move the movable devices.

### Fourth Embodiment

The fourth embodiment will be described. In this embodiment, the configuration of the first embodiment is modified as indicated below. According to this embodiment, the risk of interference is judged, and the alert level is changed in accordance with the judgment result. Parts of the structure, operation and effects that are the same as in the first embodiment will be omitted from the description.

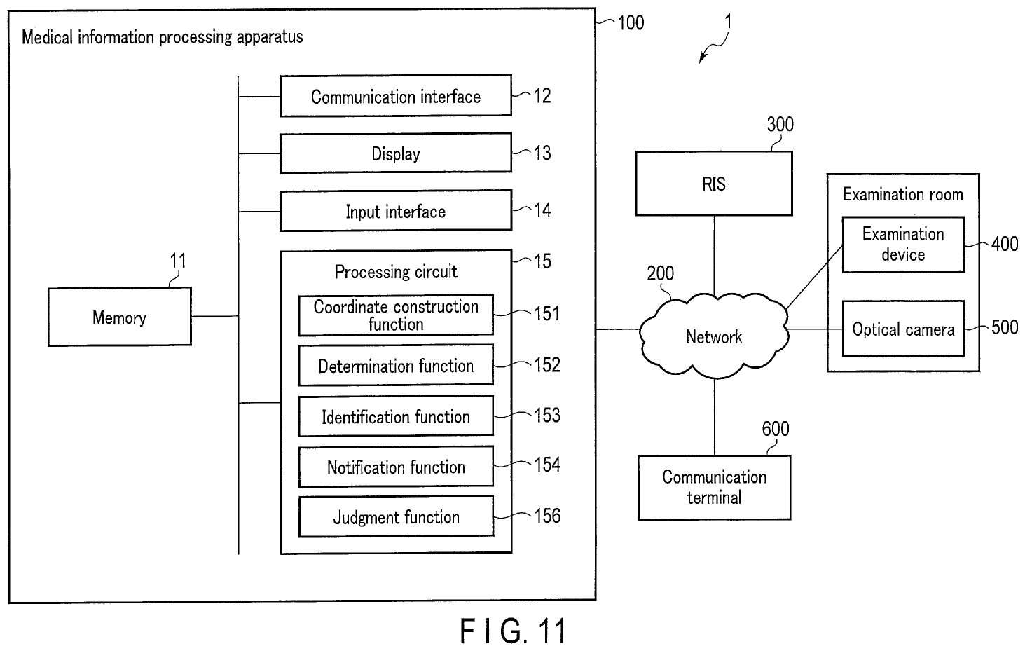

FIG. 11 is a diagram for showing the configuration of a medical information processing system 1 according to the present embodiment. According to the present embodiment, the processing circuit 15 executes a judgment function 156 in addition to the functions described in the first embodiment. With the judgment function 156, the processing circuit 15 estimates the position to which the stationary device will move during a medical examination or procedure, and judges the risk of interference (hereinafter referred to as “interference probability”) that may have been caused in the operating area of the stationary device.

The interference probability may be judged according to several preset levels, or may be judged according to a percentage between 1% to 100%. Furthermore, the interference probability may be judged on each of the sets of coordinates with respect to the 3D space coordinates constructed in the examination room, or may be judged on each of the zones obtained by dividing the 3D space coordinates of the examination room. The interference probability may also be referred to as an interference risk or interference level.

In the judgment of the interference probability, the processing circuit 15 uses, for example, the position which the stationary device is moving to (hereinafter referred to as a “target moving position”) and the direction of the moving of the stationary device. The target moving position is, for example, the position of the C-arm at X-ray imaging. The target moving position may be estimated based on the examination protocol and the information of the stationary device moving during an examination. If the target moving position or moving direction of the stationary device is unknown, the interference probability may be set in accordance with the examination protocol and operator's features.

With the notification function 154, the processing circuit 15 reports the detection result according to a notification method corresponding to the interference probability. For instance, the voice message and volume of the notification across the examination room may be changed in accordance with the interference probability. Alternatively, the display on the communication terminal 600 may be changed in accordance with the interference probability. Furthermore, in combination with the second embodiment, the amount of light on the floor surface of the examination room and/or its color may be changed in accordance with the interference probability.

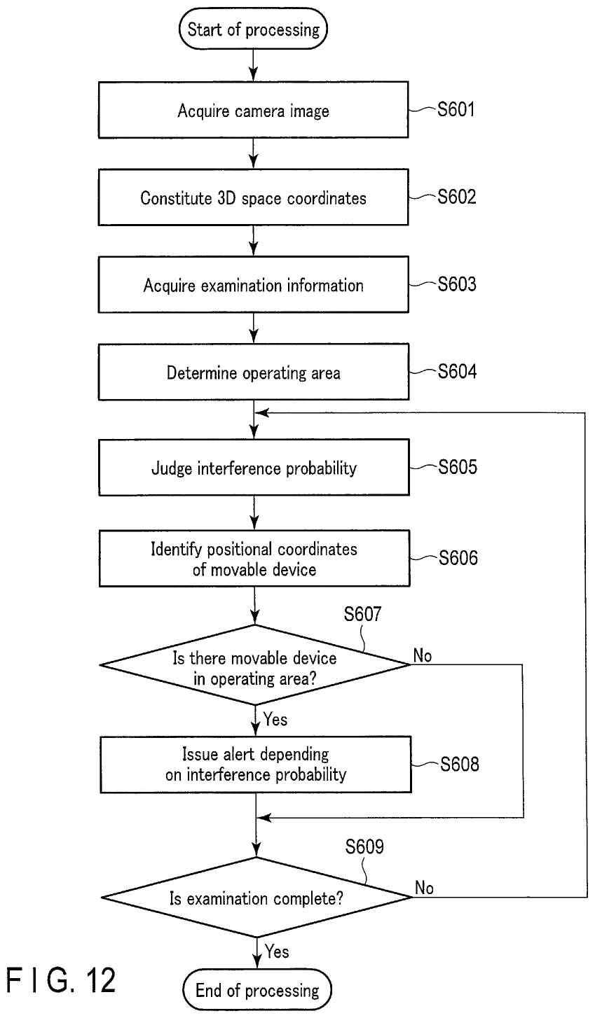

Next, the notification processing operation executed by the medical information processing system 1 will be described. FIG. 12 is a flowchart for showing an exemplary procedure of the notification process performed by the processing circuit 15 according to the present embodiment. The operations at steps S601 to S604, S606 to S607, and S609 are the same respectively as steps S101 to S106 and S108 in FIG. 4, and the explanation thereof is omitted.

### Image Generation Process

### Step S605

After the identification of the operating area of the stationary device at step S604, the processing circuit 15 judges, with the judgment function 156, the interference probability upon each position in the identified operating area. An example of a three-level interference probability, “high”, “medium”, and “low”, will be described here.

First, the processing circuit 15 receives an operation signal of the stationary device manipulated by the operator using a joystick or the like, and identifies the target moving position of the stationary device. If the imaging position or imaging angle is determined by the examination protocol, the target moving positions of the C-arm and table can be identified in advance. The processing circuit 15 judges that the risk of interference with an obstacle is high in the region in proximity to the identified target moving position, and therefore sets the interference probability to “high” for the region of the operating area of the stationary device in proximity to the target moving position.

Furthermore, the processing circuit 15 identifies the current moving direction of the stationary device based on the operation signal of the stationary device, judges that the risk of interference with an obstacle is somewhat high for the region (pass-through region) through which the stationary device passes when moving in the identified moving direction, and sets the interference probability to “medium” for the pass-through region of the operating area of the stationary device.

The processing circuit 15 judges that the risk of interference with an obstacle is low in the region other than the proximity to the target moving position and the pass-through region, and therefore sets the interference probability to “low” for a region of the operating area of the stationary device other than the proximity to the target moving position and the pass-through region.

### Step S608

If the positional coordinates of the movable device are included in the positional coordinates of the operating area of the stationary device (yes at step S607), with the notification function 154, the processing circuit 15 reports the detection result according to a notification method that corresponds to the interference probability determined at step S605. For instance, if a movable device is placed in a “low” interference probability region of the operating area of the stationary device, an alert may be sounded with a low volume, while if a movable device is placed in a “high” interference probability region, an alert message may be sent in addition to a large-volume alert sound.

The processing circuit 15 repeats the processing of steps S605 through S609 until the examination is complete. In this manner, the processing circuit 15 identifies the positional coordinates of the movable devices every time a new camera image is acquired, and if a movable device is placed in the operating area of the stationary device, an alert is issued to the people in the examination room. Furthermore, every time the operation information of the stationary device is acquired, the interference probability in the operating area is re-judged and reported according to a method corresponding to the interference probability during the examination.

The effects of the medical information processing system 1 and medical information processing apparatus 100 according to the present embodiment will be described below.

The medical information processing apparatus 100 of the medical information processing system 1 according to the present embodiment judges the interference probability for the operating area of the stationary device, and reports the detection result in accordance with a notification method corresponding to the interference probability. For instance, the interference probability is judged based on the moving direction of the stationary device and target moving position. The operator can thereby ascertain the probability of interference among the devices and take suitable measurements depending on the circumstances.

According to at least one embodiment described above, a device having a predetermined operating area can avoid interference with an obstacle.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising a processing circuit, the processing circuit being configured to:
determine an operating area relating to a device having a predetermined movable range,
identify positional coordinates corresponding to the operating area in an examination room and positional coordinates of an obstacle in the examination room, based on a camera image taken by an optical camera installed in the examination room; and
report a detection result if the positional coordinates of the obstacle are included in the positional coordinates of the operating area.

2. The medical information processing apparatus according to claim 1, wherein
the processing circuit determines the operating area in accordance with an examination protocol.

3. The medical information processing apparatus according to claim 1, wherein
the processing circuit determines the operating area in accordance with operational predispositions of an operator.

4. The medical information processing apparatus according to claim 1, wherein
the processing circuit identifies identification information of the obstacle based on obstacle information prestored in a storage medium, and
if the positional coordinates of the obstacle are detected as being included in the positional coordinates of the operating area, the processing circuit reports the identification information of the obstacle in addition to the detection result.

5. The medical information processing apparatus according to claim 1, wherein
the obstacle includes a person present in the examination room and a movable device freely movable around the examination room.

6. The medical information processing apparatus according to claim 1, wherein
the processing circuit causes a projection device arranged in the examination room to project the operating area.

7. The medical information processing apparatus according to claim 1, wherein
the processing circuit judges a moving state of the obstacle based on the camera image, and reports the detection result if the obstacle is staying still and the positional coordinates of the obstacle are included in the positional coordinates of the operating area.

8. The medical information processing apparatus according to claim 1, wherein
the processing circuit identifies positional coordinates of persons present in the examination room, and, if a movable device freely movable around the examination room is detected as the obstacle, and positional coordinates of the movable device are included in the positional coordinates of the operating area, the processing circuit informs a person in proximity to the movable device of the detection result.

9. The medical information processing apparatus according to claim 1, wherein
the processing circuit judges an interference probability of the operating area and reports the detection result in accordance with a notification method corresponding to the interference probability.

10. The medical information processing apparatus according to claim 9, wherein
the processing circuit judges the interference probability based on a target moving position or a moving direction of the device.

11. A medical information processing system comprising a processing circuit configured to:
determine an operating area relating to a device having a predetermined movable range,
identify positional coordinates corresponding to the operating area in an examination room and positional coordinates of an obstacle in the examination room, based on a camera image taken by an optical camera installed in the examination room; and
report a detection result if the positional coordinates of the obstacle are included in the positional coordinates of the operating area.

12. The medical information processing system according to claim 11, wherein
the processing circuit determines the operating area in accordance with an examination protocol.

13. The medical information processing system according to claim 11, wherein
the processing circuit determines the operating area in accordance with operational predispositions of an operator.

14. The medical information processing system according to claim 11, wherein
the processing circuit identifies identification information of the obstacle based on obstacle information prestored in a storage medium, and
if the positional coordinates of the obstacle are detected as being included in the positional coordinates of the operating area, the processing circuit reports the identification information of the obstacle in addition to the detection result.

15. The medical information processing system according to claim 11, wherein
the obstacle includes a person present in the examination room and a movable device freely movable around the examination room.

16. The medical information processing system according to claim 11, wherein
the processing circuit causes a projection device arranged in the examination room to project the operating area.

17. The medical information processing system according to claim 11, wherein
the processing circuit judges a moving state of the obstacle based on the camera image, and reports the detection result if the obstacle is staying still and the positional coordinates of the obstacle are included in the positional coordinates of the operating area.

18. The medical information processing system according to claim 11, wherein
the processing circuit identifies positional coordinates of persons present in the examination room, and, if a movable device freely movable around the examination room is detected as the obstacle, and positional coordinates of the movable device are included in the positional coordinates of the operating area, the processing circuit informs a person in proximity to the movable device of the detection result.

19. The medical information processing system according to claim 11, wherein
the processing circuit judges an interference probability of the operating area and reports the detection result in accordance with a notification method corresponding to the interference probability.

20. The medical information processing system according to claim 19, wherein
the processing circuit judges the interference probability based on a target moving position or a moving direction of the device.
